(19) United States
(12) Patent Application Publication
(10) Pub. No.: US 2014/0299132 A1
(43) Pub. Date: Oct. 9, 2014

(54) SYSTEMS AND METHODS FOR PROVIDING POSITIVE AIRWAY PRESSURE IN A TUBE-LIKE STRUCTURE

(71) Applicants: Kevin Scott Librett, Watertown, MA (US); Karl R Leinsing, Dover, NH (US)

(72) Inventors: Kevin Scott Librett, Watertown, MA (US); Karl R Leinsing, Dover, NH (US)

(73) Assignee: HUMAN DESIGN MEDICAL, LLC, Charlottesville, VA (US)

(21) Appl. No.: 14/216,882

(22) Filed: Mar. 17, 2014

Related U.S. Application Data
(60) Provisional application No. 61/798,367, filed on Mar. 15, 2013.
(60) Provisional application No. 61/798,541, filed on Mar. 15, 2013.
(60) Provisional application No. 61/798,462, filed on Mar. 15, 2013.
(60) Provisional application No. 61/798,263, filed on Mar. 15, 2013.

(51) Int. Cl. A61M 16/00; A61M 16/06; A61M 16/08

(52) U.S. Cl. CPC A61M 16/0066; A61M 16/0816; A61M 16/06; A61M 16/0051; A61M 16/0003; A61M 2016/003
USPC 128/205.25; 128/204.18

(57) ABSTRACT

The systems and methods described herein include a small system integrated within a tubing for providing positive air pressure to a patient. The system may include a tube with an inlet opening, an expansion section with a gas flow generator, and an outlet opening. The systems and methods described herein provide improved mobility, comfort, and usability for patients.

### CROSS-REFERENCE TO RELATED APPLICATIONS

This application claims the benefit of U.S. Patent Application 61/798,367 filed on Mar. 15, 2013; U.S. Patent Application 61/798,541 filed on Mar. 15, 2013; U.S. Patent Application 61/798,263 filed on Mar. 15, 2013; and U.S. Patent Application 61/798,462 filed on Mar. 15, 2013 which are incorporated herein by reference.

### COPYRIGHT INFORMATION

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention relates to a positive airway pressure [PAP] devices, such as continuous positive airway pressure [CPAP] devices, and more particularly to a PAP device mounted or formed within a tube or hose.

### BACKGROUND OF THE INVENTION

The sleep apnea syndrome afflicts an estimated 1% to 5% of the general population and is due to episodic upper airway obstruction during sleep. Those afflicted with sleep apnea experience sleep fragmentation and intermittent, complete, or nearly complete cessation of ventilation during sleep with potentially severe degrees of oxyhemoglobin desaturation.

It is known that applying a positive airway pressure to a patient with a CPAP device may prevent upper airway occlusion during sleep. CPAP devices have become the apparatus of choice for the treatment of chronic sleep apnea, chronic pulmonary obstruction and snoring. Many CPAP machines are readily available in the marketplace.

A typical CPAP system generally includes a bedside generator comprising, a blower unit powered by an electric motor. The blower unit, the motor, and associated controls are usually encased together within the bedside generator. A delivery tube, usually a flexible plastic tube having a proximal end and a distal end, is used to deliver pressurized air or other gasses to the patient. The proximal end of the delivery tube is connected to the bedside generator and the distal end of the delivery tube is fitted to the face of a patient. The patient interface may include features that allow the patient interface to be affixed to the patient and maintain a proper orientation with respect to the patient.

Bedside CPAP machines are typically large and heavy. They are usually plugged into a wall outlet for power or have a large external battery. The size, weight, and power constraints can interfere with patients' ability and willingness to use the machine. For example, these constraints can make it difficult to utilize the CPAP apparatus in areas away from their bedside or while traveling. Additionally, these constraints can also prohibit patients from moving freely during sleep, thereby inducing further discomfort.

Furthermore, typical CPAP devices are relatively loud and can interfere with a patient's sleep or the sleep of other people nearby. In a typical CPAP device, sound may be propagated from various locations and actions of the device, such as the flow of air the flow of air into and out of the device or the operation of the motor and fan. Because the apparatus is used mainly in a bedroom or other place having a low ambient noise level to facilitate sleep, it is important that the blower operates quietly so as not to disturb the patient or others in close proximity while they sleep.

A need therefore exists for PAP devices with size, weight, and sound characteristics that provide improved usability for patients.

Many CPAP devices are large in nature that they are required to be placed on a bedside table, under the bed, or otherwise away from the patient. Attempts have been to mount some CPAP devices on a mask or top of a person's head in an effort to make CPAP devices portable, less cumbersome and more comfortable. However, in some respects these devices fail to adequately address comfort, weight, noise attenuation from the device and so forth.

### SUMMARY OF THE INVENTION

In one exemplary embodiment a long hose or tube comprises an expansion section that houses a blower configured to provide pressurized air flow. The intake portion of the hose may be configured to have a single inlet. Various replaceable filters may be placed immediately before or after the inlet of the hose.

In some embodiments the length of the hose provides a sufficient length to provide an effectively large enough chamber to reduce any attenuated noise.

In one embodiment multiple expansion chambers functioning as acoustic reduction chambers are provided.

In one embodiment a portion of the hose contains electrical leads or a power cord to supply power to the blower.

In one embodiment the exterior of the expansion portion contains a display screen and input buttons. (it is also contemplated the display screen may be a touch screen) The input buttons may be used to power on/off the CPAP in hose device and select various features.

In one embodiment flexible electrical circuitry is used in the non-expansion portion of the hose or tube.

In another embodiment a portable and elongated CPAP system comprising an expansion tube section having a blower comprised of a motor and impellor disposed therein; at least two detachable tube sections for attaching to an inlet and outlet port of the expansion tube section, wherein one of the detachable tube sections has acoustically designed inlet port that forms a low pass frequency filter with an expansion chamber disposed in the expansion tube section.

These aspects of the invention are not meant to be exclusive and other features, aspects, and advantages of the present invention will be readily apparent to those of ordinary skill in the art when read in conjunction with the following description, appended claims, and accompanying drawings.

### BRIEF DESCRIPTION OF THE DRAWINGS

The foregoing and other objects, features, and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a cross-sectional schematic view of a PAP system integrated into a hose.

FIG. 2 is an exterior view of a PAP system integrated into a hose.

FIG. 3 illustrates noise filter system.

FIG. 4A illustrates a PAP system integrated into a hose connected to a mask being worn by a user.

FIG. 4B shows a PAP system integrated into a hose with detachable segments.

FIG. 5 depicts a cross-sectional view of PAP system integrated into a hose with a reduced-diameter air inlet tube.

FIG. 6 illustrates a PAP system integrated into a hose with an air inlet tube having multiple turns.

FIG. 7 is an exterior view of a PAP system integrated into a hose having multiple expansion sections.

FIG. 8 illustrates a PAP system integrated into a hose with a reduced diameter inlet opening.

FIGS. 9A-B depict PAP systems integrated into a hose with curved or extended flow paths.

FIGS. 10A-B depict a PAP system integrated into a hose with detachable segments.

FIGS. 11A-C illustrate various silencer intake segments having varying lengths.

FIGS. 12A-B illustrate linked expansion chambers.

### DETAILED DESCRIPTION OF THE INVENTION

To provide an overall understanding of the systems, devices, and methods described herein, certain illustrative embodiments will be described. Although the embodiments and features described herein are frequently described for use in connection with CPAP apparatuses, systems, and methods, it will be understood that all the components, mechanisms, systems, methods, and other features outlined below may be combined with one another in any suitable manner and may be adapted and applied to other PAP apparatuses, systems, and methods, including, but not limited to, automatic positive airway pressure devices [APAP], variable positive airway pressure devices [VPAP], bi-level positive airway pressure devices [BPAP], and related apparatuses, systems, and methods.

Bedside CPAP machines are typically large, heavy, and noisy. The systems and methods described herein are directed towards a small, quiet, light-weight, and portable CPAP device to overcome this current limitations and disadvantages.

The systems and methods described herein provide pressurized gas to the airway of a patient. In certain embodiments, the systems and methods described herein include a gas flow generator for providing a flow of gas to a mask or other oral-nasal region device for the delivery of the gas to an airway of a patient. In certain approaches, a single hose is used with at least one expansion chamber housing a blower.

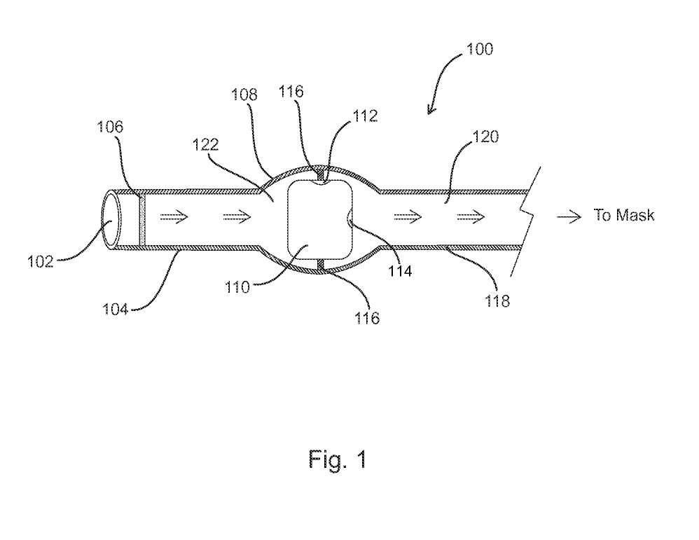

FIG. 1 is a cross-sectional schematic view of a PAP system integrated into a hose. PAP system 100 includes an upstream hose section 104, expansion chamber 108 with a blower 110, and downstream hose section 118. System 100 may include a filter 106. For example, filter 106 may be positioned in upstream portion 104. In certain approaches, upstream portion 104 is removable from expansion chamber 108. In certain approaches, downstream portion 118 is removable from expansion chamber 108.

In certain embodiments, system 100 includes a filter 106 to clean the air of particulate matter. Filter is positioned upstream of blower 110. For example, filter 106 may be positioned within upstream hose portion 104. Additionally or alternatively, filter 106 may be positioned within expansion chamber 108. In certain embodiments, filter 106 is removable so that it may be cleaned, replaced, or adapted for a particular need. For example, various types of filters may be used for filter 106 depending on a patient's health. Filter 106 may not be required for all patients, may be replaceable, or may be cleaned.

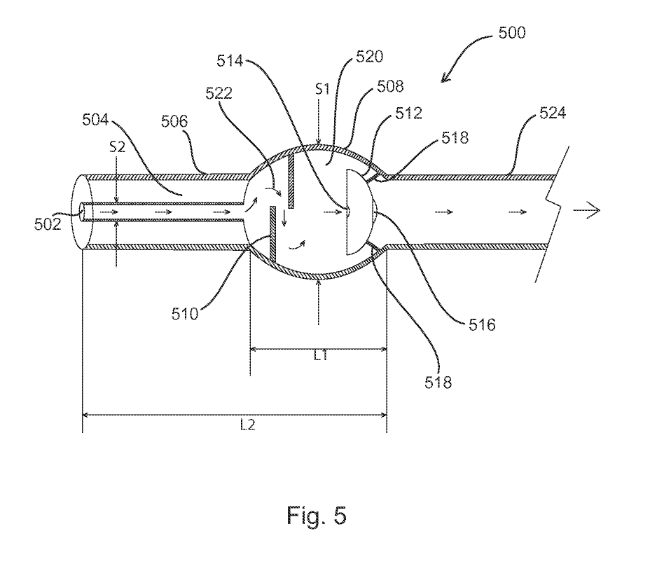

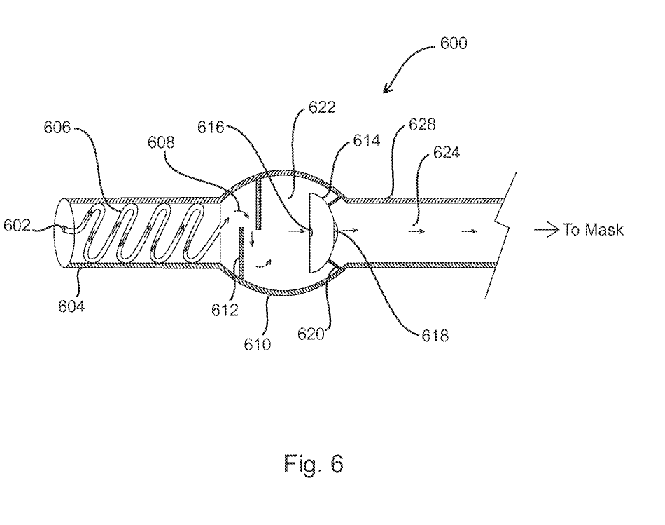

Blower 110 is positioned within the space 122 of expansion chamber 108. In certain approaches, blower 116 fills space 122. Blower 110 may have any appropriate shape, and in certain approaches may be shaped similarly as the interior space 122 of expansion camber 108. In certain approaches, blower 116 is coupled to chamber 108 with mount connects 116. In addition to connecting blower 110 to expansion chamber 108, mount connects 116 may reduce or eliminate transfer of vibrations from the blower to other components of device 100. In certain embodiments, blower 110 is a brushless air-bearing motor. Blower 110 may be an air-bearing blower or other type of blowers used in the industry, and may include various impellors that rotate sufficiently to create an internal vacuum for pulling air through intake 112 and/or increase pressure through blower output 114 to create pressurized air flow 120 through downstream hose portion 118. In some embodiments the blower is an inline or pass-through blower meaning the impellor is placed directly in the flow path. See for example FIGS. 5 and 6.

During operation, PAP device 100 creates positive air pressure through downstream hose section 118. For example, when a patient interface, such as a mask, is attached, PAP device 100 creates positive air pressure, which can be provided to the patient when the patient places the patient interface at his or her airways (e.g., nose or mouth). Blower 110 includes intake 112. When blower 110 is powered on, blower 110 intakes air through intake 112 and pushes out that air through outlet 114. The reduced pressure at intake 112 causes air to flow through inlet 102, through filter 106, through upstream hose portion 104, and into chamber 108, where the air then flows into intake 112 of blower 110. Blower 110 then pushes air through outlet 114, and through downstream hose portion 118 to provide positive air pressure, for example, through a mask. In certain approaches, the pressurized air is delivered at a pressure ranging from approximately 2 centimeters (cm) of water to approximately 40 cm of water above atmospheric pressure at the point of use, although any appropriate pressure may be used.

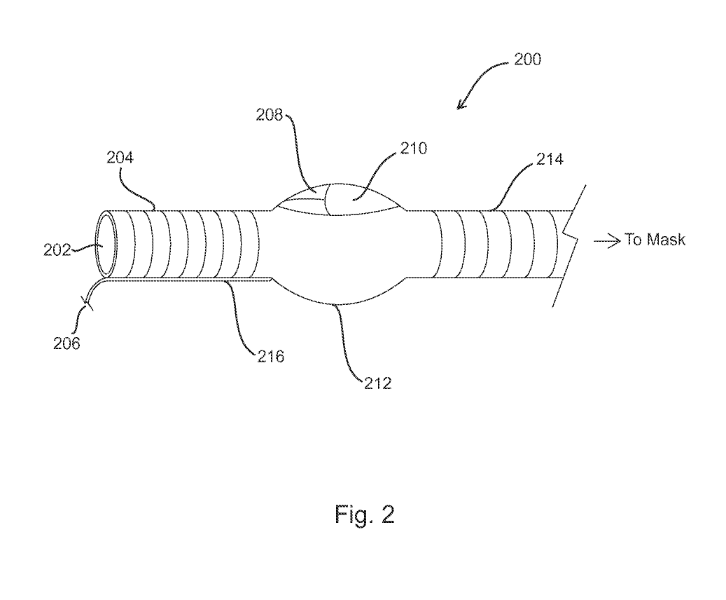

Apparatus 100 may also include a pressure port. The pressure port may formed inline with the tubing downstream from the blower and run adjacent the tube into chamber 122, where the pressure port couples to a pressure sensor, such as a pressure sensor on a circuitry board. The pressure port provides fluid communication from the output of device 100 to a pressure sensor coupled to control circuitry. The circuitry board may include control circuitry and control components for the operation of device 100. The circuitry board may be positioned in the expansion section, remotely, embedded along the length of the tube and so forth. In certain approaches, the circuitry board includes a power sources, such as a power adapter or battery. In certain approaches, the control circuitry on board of device 100 is configured to display the pressure measured through pressure port at a display, such as display 210 depicted in FIG. 2. In certain embodiments, the pressure output of device 100 may be adjusted manually by the user with user interface buttons. In certain approaches, the control circuitry on board is configured to automatically adjust the output of device 100 based on the pressure measurements. The output of device 100 may be adjusted by modulating the power of blower 110.

FIG. 2 is an exterior view of a PAP system integrated into a hose. PAP system 200 includes an upstream hose section 204, expansion chamber 212 where a blower (not depicted) is positioned, and downstream hose section 214. System 200 includes a power cable 206. In certain approaches, cable 206 is coupled to the exterior surface 216 of system 100. Additionally or alternatively, power cable 206 may be positioned on the interior of system 100. In certain approaches, upstream portion 204 is removable from expansion chamber 212. In certain approaches, downstream portion 214 is removable from expansion chamber 212.

System 200 includes user controls components, such as interface buttons 208 and display 210 for controlling and using apparatus 200. For example, a user may be able to turn the power on and off, adjust pressure settings, set a timer, run system diagnostic tests, and control or adjust other functions. Display 210 may be any appropriate display, including, but not limited to an LED or LCD display. Although 1-3 user interface buttons 208 are depicted, any appropriate number of buttons may be used. In certain approaches, a PAP apparatus, such as system 200, may include between 1 and 10 user interface buttons. In certain approaches, user interface buttons are included in display 210. For example, display 210 may be a capacitive or pressure sensitive touch screen display. Further, buttons 208 and display 210 may vary in size between different embodiments. For example, some embodiments may include a larger display, while other embodiments may include a smaller display. Display 210 may display data or control functions, such as pressure levels, time, use time, or other information. Display 210 may show one piece of data or function or a plurality of data and functions.

The hose or tubing (such as upstream portion 204, chamber 212, and downstream portion 214) used in system 200 may be flexible and provide the ability to somewhat conform to the movements and body of a user. In certain approaches, a user may be sleeping on his/her side or back and the system 200 is sufficiently light and flexible so that system 200 rotates or follows the rotation or turning of the user's body. In some embodiments (not shown in the figures) a battery source is contained within system 200.

During operation, PAP device 200 creates positive air pressure through downstream hose section 214. System 200 pulls air through opening 202 of upstream hose section 204, through section 204, into chamber 212, and through downstream section 214. For example, when a patient interface, such as a mask, is attached, PAP system 200 creates positive air pressure, which can be provided to the patient when the patient places the patient interface at his or her airways (e.g., nose or mouth).

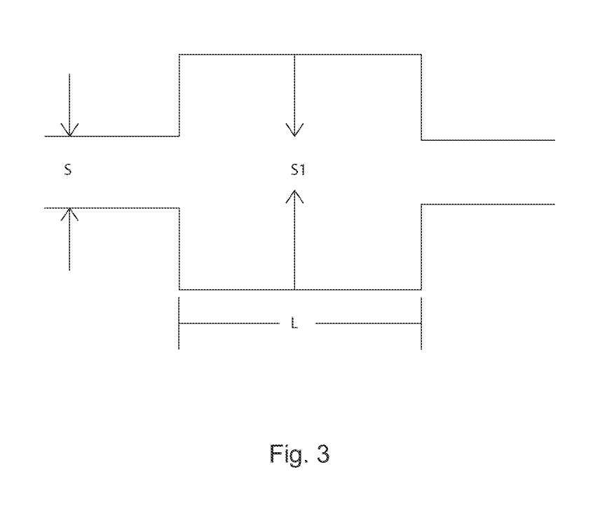

FIG. 3 illustrates a low-pass acoustic filter system. The equation below describe the effects modifying each geometrical section of the filter system has on the system.

$${T}_{π} = ( \frac{1}{1 + ( \frac{{S}_{1} - S}{2 S} ) kL} ) \qquad Equation 1$$

In Equation 1, T is the power transmission, also referred to as the acoustic output, sound level, or noise level; k is the wavenumber of the sound; S1 is the area of an expansion chamber; L is the length of an expansion chamber; and S is the area of an inlet port or tube. Thus, if S1 increases in size, L increases in length or S decreases in area, then the power transmission T is reduced.

In accordance with the present disclosure, the area of the respective expansion chamber (S1) and the area of its inlet port (S) may have a proportional relationship. For example, the area of the chamber may be larger than the area of the inlet port by a factor of 2. In additional embodiments, S1 may be larger than S by a factor ranging from a factor of approximately 2 to a factor of approximately 20 or more. In at least one embodiment, S1 is larger than S by a factor of about 10. Additionally, the length of L may be increased wherein the upstream portion of the tube and the expansion chamber effectively act as single chamber, thus decreasing the amount of noise emanating from the system.

Referring to FIG. 3, the inlet pathway defined by S is smaller than the upstream portion of the expansion chamber. In accordance with equation 1, when S is reduced relative to S1, then T or the noise level is attenuated. By increasing L (the length of the expansion chamber), the noise may be further attenuated. In addition, if the inlet pathway is sufficiently long, the effective length of the expansion chamber increases from L to L1, thus also reducing the noise of the system. An increase in length of intake tubes helps decrease the amount of noise escaping the system. Thus, longer inlet or sections of tubes help attenuate the noise of the system.

There exists a proportional relationship between the length of the inlet tube or port and the cross-sectional area of the inlet port with the volume (and length) of the receiving acoustic chamber or expansion section/chamber. However, by increasing the length of the inlet port and restricting the cross-sectional area of the inlet port may cause increased resistance to air flow in the system. This may in turn cause a blower disposed inside an acoustic chamber to have to work harder, which may result in an increase in noise generation from the blower (and motor of the blower). Thus, a balancing and optimization step is often required when trying to create a sufficiently portable PAP device that is both quiet and small in size. Equation 2, illustrates this relationship of increasing modifying the various dimensions of the inlet port and the effect it has on the increased motor work and noise.

$$Resistance of air flow ∝ \frac{Length inlet}{Area inlet} ∝ Motor Work ∝ Motor Noise \qquad Equation 2$$

Another way of describing this is a smaller inlet diameter increases air flow resistance, which increases motor noise. Some practical steps have been incorporated to also position inlet ports on the PAP device such that they point away from the ears of the user. For example, in several of the figures the inlet port is on the opposite end of the outlet port and adapters, which lead to the tubing that takes air to the mask placed over the user's nose and/or mouth. In several instances most of the noise escaping the system leaves through the inlet port.

In one preferred embodiment the tube has a diameter of approximately 1.0″ and an inlet port has a diameter of ⅜″ that leads into a larger volume. In another embodiment the ⅜″ in diameter inlet port folds back or bends creating additional length and noise attenuation. However, as mentioned it is important to balance this with any increase in work to motor of the blower, which may result in increased noise generation. Inline blowers may be placed substantially down the length of the tubing leading to the mask.

Equation 1 can also be used to describe the relationship between length and noise attenuation in an individual tube. In the case of a single, individual tube, S1 is equal to S. Accordingly, the noise output T is reduced when the tube is lengthened (L is increased). This characteristic is important because the length of the intake tube (such as intake tube 115) can be used to decrease the noise of the PAP device (such as device 100 and other systems and methods described herein).

Equation 3 describes the relationship between the cut-off frequency of the acoustic filtering and the length and areas of the chamber and tube:

$${f}_{c} = ( \frac{Sc}{π L ( {S}_{1} - S )} ) \qquad Equation 3$$

In equation 3: f_{c} is the cutoff frequency; c is the speed of sound; S1 is the area of the expansion chamber; L is the length of the tube or chamber; and S is the area of inlet port. Thus, as L or S1 become larger in value, and/or S becomes smaller, the cutoff frequency becomes lower and every frequency above the cutoff frequency is significantly attenuated. In practical terms, the cutoff frequency f_{c} can be reduced by increasing the ratio of S1:S, for example by decreasing the area of the inlet and/or increasing the area of the expansion chamber. Additionally, lengthening the expansion chamber (increase L) will also reduce the cutoff frequency.

The length of an intake tube may range from approximately 1 inch to approximately 8 inches or longer. In accordance with FIG. 3, equation 1, and equation 2, the length and diameter of the intake tube may be adjusted to affect the overall noise attenuation of the PAP device.

In order to maximize the length of the intake tube so as to further attenuate the noise of the device, the tube may be angled, have one or more bends or turns in any 3-dimensional direction, or it may have a spiral-like configuration. Similarly, interchamber tubes and outlet tubes may also include bends, turns, angles, spirals, or other configurations.

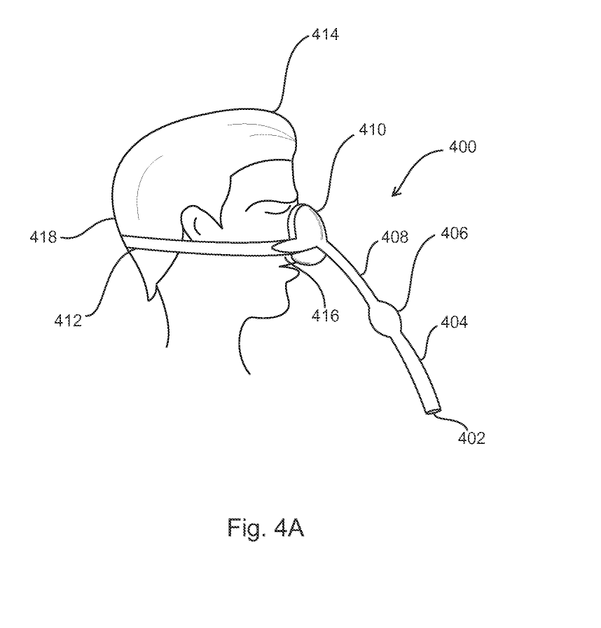

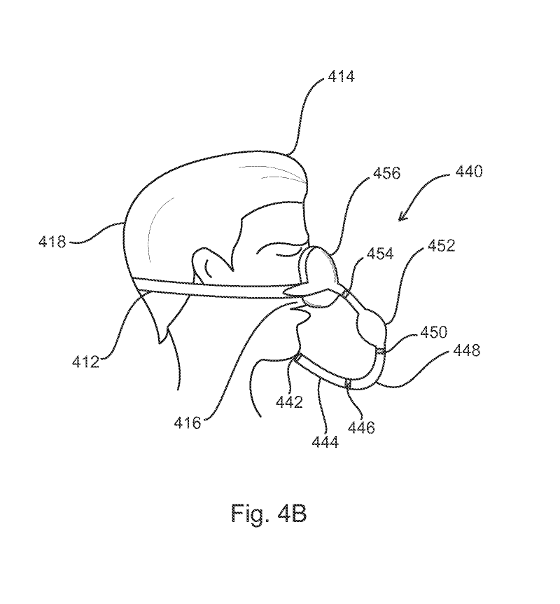

FIG. 4A illustrates a PAP system integrated into a hose connected to a mask being worn by a user. PAP system 400 includes an upstream hose section 404, expansion chamber 408 with a blower (not depicted), and downstream hose section 408 coupled to a user mask 410. Mask 410 is coupled to the respiratory pathways 416 of user 414. A strap 412 is integrated with mask 410 and wraps around the head 418 of user 414. In certain approaches, upstream portion 404 is removable from expansion chamber 406. In certain approaches, downstream portion 408 is removable from expansion chamber 406.

During operation, PAP device 400 creates positive air pressure to the respiratory pathways 416 through mask 410. An internal blower positioned within chamber 406 of system 400 pulls air through opening 402, through section 404, into chamber 406, through downstream section 408, and into mask 410.

FIG. 4B shows a PAP system integrated into a hose with detachable segments. PAP system 440 includes a first upstream hose section 444 with opening 442, second upstream hose section 448, chamber 452, and mask 456. Expansion chamber 456 includes an internal blower (not depicted). Mask 456 is coupled to the respiratory pathways 416 of user 414. A strap 412 is integrated with mask 456 and wraps around the head 418 of user 414.

In certain approaches, section 444, section 448, chamber 452, and mask 454 are removably coupled. Although two hose sections are depicted, any number of sections may be used. Additionally or alternatively, hose sections may also be used downstream of chamber 452. System 440 also includes coupler 446 to join first upstream hose section 444 with section upstream hose section 448, coupler 450 to join second upstream hose section 448 with chamber 452, and coupler 454 to join chamber 452 with mask 456. Hose section 444 and hose section 448 may be curved. For example, as depicted, hose section 444 and 448 may be curved to reduce the overall length of system 440 to make it more convenient and easier to move with. In certain embodiments, the hoses sections, and specifically, the intake hole 442, or directed away from the ears of user 414 to minimize noise heard by the operation of system 440.

FIG. 5 depicts a cross-sectional view of PAP system integrated into a hose with a reduced-diameter air inlet tube. PAP system 500 includes an upstream hose section 506 with an intake 502 having a reduced diameter S₂. As described in relation to FIG. 3, equation 1, and equation 2, a reduced diameter intake can attenuate the noise of operation, thereby making the operation and use of system 500 quieter and more comfortable for the user. Hose section 506 may also include closed portion 504, which has no airlow. In certain approaches, section 504 contains other parts of system 500, such as a cables or wire for powering or controlling system 500. Expansion chamber 508 is effectively hermetically sealed with only the intake 502 and downstream section 524 allowing for fluid to enter and escape.

Hose section 504 leads to expansion chamber 508 with a blower 512. In certain approaches, blower 512 is coupled to chamber 508 with mount connects 518. In addition to connecting blower 512 to expansion chamber 508, mount connects 518 may reduce or eliminate transfer of vibrations from the blower to other components of device 500. Expansion chamber 508 may include attenuators 510. For purposes of the systems and methods described herein, an attenuator may refer to any of a plane, bar, circular, semi-circular, sphere, cone, or other mechanism configured to deflect, absorb, weaken and/or reduce a sound wave. Although two attenuators 510 are depicted in FIG. 5, any number of attenuators may be used. The attenuators 510 create an extended airflow path 522, which reduces the noise of the system. The air flows through space 520 and into the inlet 514 of blower 512, which then blows air through outlet 515 into section 524.

Expansion chamber 508 has a first length of L₁ and an area depicted by S₁. The length of hose section 506 and chamber 508 is indicated by L₂. These representative dimension are analogous to those discussed in relation to FIG. 3.

In certain approaches, upstream portion 506 is removable from expansion chamber 108. In certain approaches, downstream portion 118 is removable from expansion chamber 108.

FIG. 6 illustrates a PAP system integrated into a hose with an air inlet tube having multiple turns. PAP system 600 includes an upstream hose section 604 with an intake 602 having a reduced diameter. As described in relation to FIG. 3, equation 1, and equation 2, a reduced diameter intake can attenuate the noise of operation, thereby making the operation and use of system 600 quieter and more comfortable for the user.

Hose section 604 may include air pathway 606. As shown, air pathway 606 has multiple turns thereby extending the total air pathway length leading to expansion chamber 610. The bends and turns in air pathway 606 help prevent noise from escaping the CPAP in a tube system. Pathway 606 may spiral internally inside the upstream portion 604. Although pathway 606 is depicted as internal within portion 604, in certain approaches, portion 604 itself can spiral or turn, for example, in a fashion similar to a telephone cord. Hose section 604 leads to expansion chamber 610 with a blower 614. In certain approaches, blower 614 is coupled to chamber 610 with mount connects 620. In addition to connecting blower 614 to expansion chamber 610, mount connects 620 may reduce or eliminate transfer of vibrations from the blower to other components of device 600.

Expansion chamber 610 may include attenuators 612. Although two attenuators 612 are depicted in FIG. 6, any number of attenuators may be used. The attenuators 612 create an extended airflow path 608, which reduces the noise of the system. The air flows through space 622 and into the inlet 616 of blower 614, which then blows air through outlet 618 into section 628, where it then flows along path 624 for delivery to a patient.

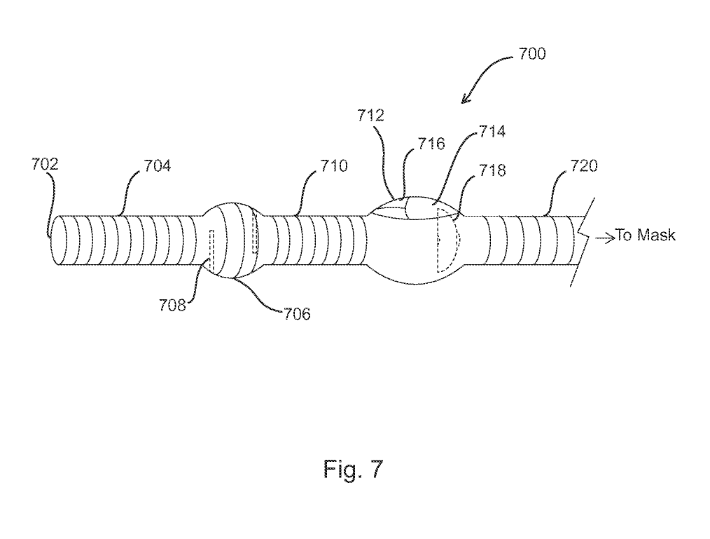

FIG. 7 is an exterior view of a PAP system integrated into a hose having multiple expansion sections. PAP system 700 includes a first upstream hose section 704 with opening 702, first expansion chamber 706, second upstream hose section 710, expansion chamber 712 where blower 718 is positioned, and downstream hose section 712. In certain approaches, sections 704, 710, 720 and chamber 706 and 712 are removably coupled. In certain approaches, system 700 includes attenuators, such as attenuators 708 positioned in chamber 706 for reducing noise output of system 700. Although attenuators 708 are depicted in chamber 706, they may be positioned in any portion of system 700, including, first upstream hose section 704, chamber 706, second upstream hose section 710, chamber 712, and downstream section 720.

System 700 includes user controls components, such as interface buttons 716 and display 714 for controlling and using apparatus 700. For example, a user may be able to turn the power on and off, adjust pressure settings, set a timer, run system diagnostic tests, and control or adjust other functions. Buttons 716 and display 714 may be similar to previously described buttons 208 and display 210.

The expansion chambers 706 and 712 serve to reduce the noise output of system 700. As previously described in relation to FIG. 3 and equation 1 and equation 2, the tubing and expansion chambers form noise filters. Expansion chambers 706 and 712 may include anechoic or dissipative materials to further reduce the sound levels. Dissipative elements may include anechoic materials such as foam, rubber, clay, silicon, or any other suitable soft and/or porous materials. Although two expansion chambers are depicted, any appropriate number of chambers may be used.

During operation, PAP device 700 creates positive air pressure through downstream hose section 720. System 700 pulls air through opening 702 of first upstream hose section 704, through section 704, through chamber 706 through second upstream hose section 704, and into chamber 712. Blower 718 then blows the air through downstream section 720, which can be provided to the patient when the patient places the patient interface (e.g., mask) at his or her airways (e.g., nose or mouth).

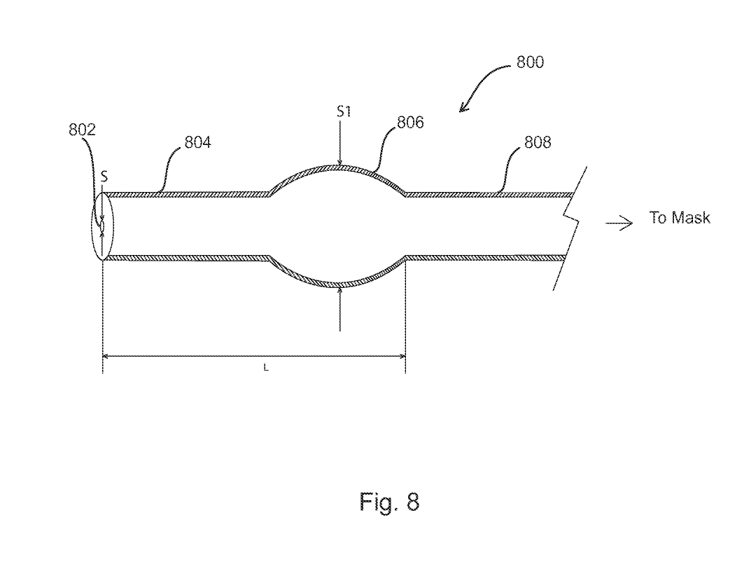

FIG. 8 illustrates a PAP system 800 integrated into a hose with a reduced diameter inlet opening 802. Inlet opening 802 of upstream hose portion 804 has an opening area S, which is smaller than the general diameter of upstream hose portion 804. Inlet opening 802 immediately opens or expands into the larger internal diameter of the tube, which leads into expansion chamber 806 with an area 51. In this example, upstream hose portion 804 acts as a first expansion chamber for purpose of the noise-pass filter equations and expansion chamber 806 acts as a second expansion chamber. Alternatively, upstream hose portion 804 and expansion chamber 806 may effectively be calculated as a single chamber with increased area, where L becomes the length of the inlet 802 to the end of expansion chamber 806 where the positive air pressure is formed exiting from the blower (not depicted) into downstream hose portion 808. Each interface with a size change provides an opportunity to create a frequency cutoff system or low power transmission region.

Although the hosing and tubing is generally depicted as round, any appropriate shape may be used. For example, a square cross-sectional shaped hose or tube may be used.

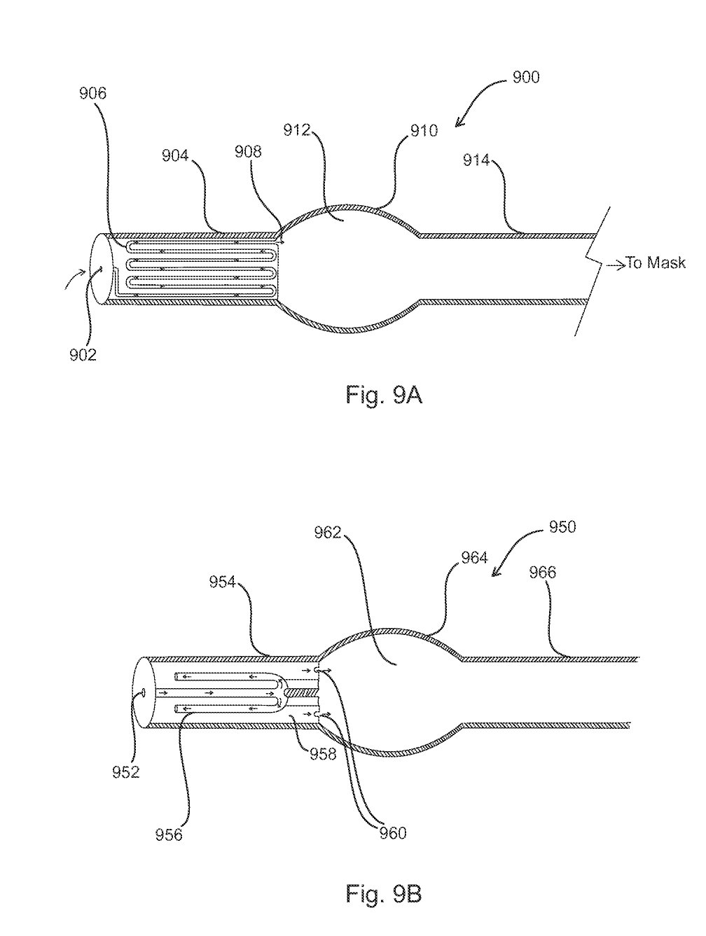

FIGS. 9A-B depict PAP systems integrated into a hose with curved or extended flow paths. In FIG. 9A, PAP system 900 includes an upstream hose section 904 with a reduced diameter intake 902. As described in relation to FIG. 3, equation 1, and equation 2, a reduced diameter intake can attenuate the noise of operation, thereby making the operation and use of system 600 quieter and more comfortable for the user.

Hose section 904 includes air pathway 906. As shown, air pathway 906 has multiple turns thereby extending the total air pathway length leading to expansion chamber 910. The bends and turns in air pathway 906 help prevent noise from escaping the CPAP in a tube system. Pathway 906 may spiral internally inside the upstream portion 905. Hose section 904 leads to expansion chamber 910 with a blower (not depicted). In certain approaches, expansion chamber 910 may include attenuators, similar to attenuators 612. The air flows through space 912 and is blown into section 914, where it then can be delivered to a patient.

FIG. 9B illustrates an additional embodiment with a curved intake path. PAP system 950 includes an upstream hose section 954 with a reduced diameter intake 952 leading to air pathway 956. As shown, air pathway 956 has multiple turns thereby extending the total air pathway length leading to expansion chamber 964. The bends and turns in air pathway 956 help prevent noise from escaping the CPAP in a tube system. Pathway 956 may have sections that split into other portions of section 954, such as inner portion 958. For example, pathway 956 may be a curved tube within hose section 954 that has an open end positioned within inner portion 958. Hose section 904 leads to expansion chamber 964 with a blower (not depicted). For example, hose section 954 has outlets 960 from inner portion 958 into chamber 964. In certain approaches, expansion chamber 964 may include attenuators, similar to attenuators 612. The air flows through space 962 and is blown into section 966, where it can then be delivered to a patient.

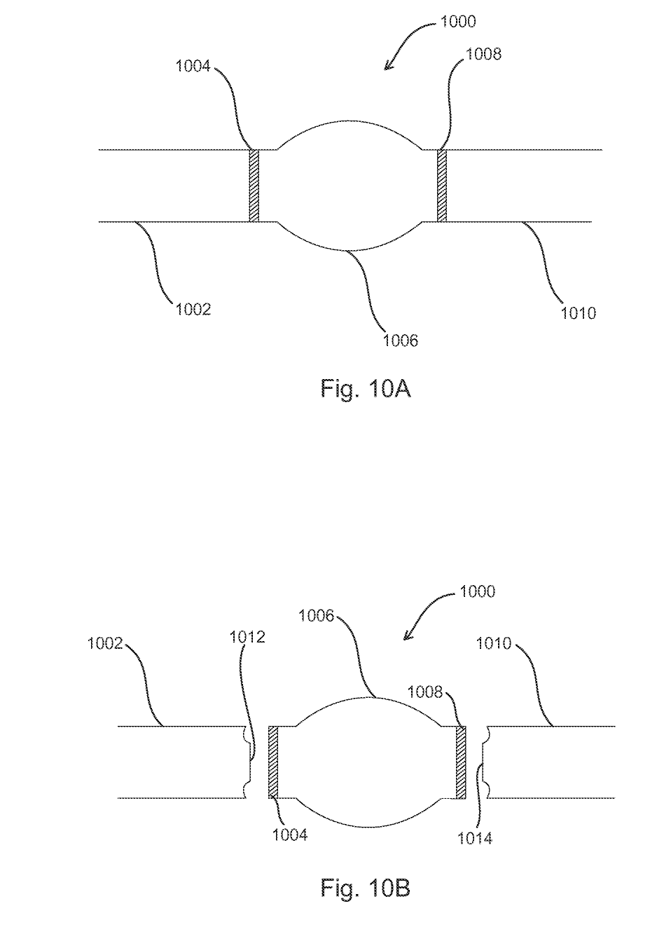

FIGS. 10A-B depict a PAP system integrated into a hose with detachable segments. System 1000 includes a first hose 1002, expansion chamber 1006, and second hose 1010. First hose 1002 is removably coupled to expansion chamber 1006 with coupler 1004. Similarly, second hose 1010 is removably coupled to expansion chamber 1006 with coupler 1008. End 1012 of hose 1002 is coupled to coupler 1004. Similarly, end 1014 of hose 1010 is coupled to coupler 1008. Although coupler 1004 and coupler 1008 are shown in FIG. 10B as attached to chamber 1006, other configurations are possible. In certain approaches, at least one of coupler 1004 and coupler 1008 are permanently coupled to chamber 1006. In certain approaches, coupler 1004 is permanently coupled to hose 1002. In certain approaches coupler 1008 is permanently coupled to hose 1010. In certain approaches coupler 1004 is a separate component that is removably coupled to both hose 1002 and chamber 1006. Similarly, in certain approaches coupler 1008 is a separate component that is removably coupled to both hose 1010 and chamber 1006. Coupler 1004 and coupler 1008 may be a compression fitting, threaded fitting, push-fit fitting, friction fit fitting, or any other appropriate fitting or coupling mechanism.

In certain approaches, the systems and methods described herein can be adaptable to accommodate different tube lengths to meet patient requirements for noise levels, comfort, and size. For example, a longer intake tube acts as a better noise attenuator than a shorter intake tube.

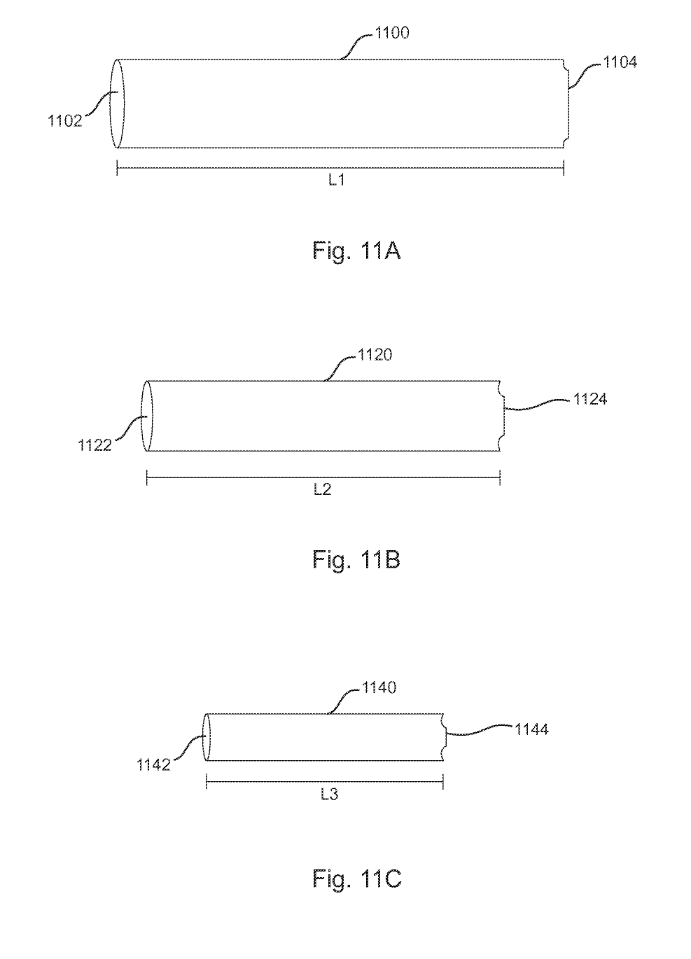

FIG. 11A illustrates a first hose 1100, with an intake end 1102 and a coupling end 1104. First hose 1100 has a length of L1. Coupling end 1104 may be coupled to an expansion chamber with a coupler, for example, to expansion chamber 1006 with coupler 1004. FIG. 11B illustrates a second hose 1120, with an intake end 1122 and a coupling end 1124. Second hose 1120 has a length of L2, which is shorter than L1. Coupling end 1124 may be coupled to an expansion chamber with a coupler, for example, to expansion chamber 1006 with coupler 1004. FIG. 11C illustrates a third hose 1140, with an intake end 1142 and a coupling end 1144. Third hose has a length of L3, which is shorter than L2. Coupling end 1144 may be coupled to an expansion chamber with a coupler, for example, to expansion chamber 1006 with coupler 1004.

These hoses represent, from a general principle, different sound properties when used with the PAP systems and devices described herein. In general, a longer intake tube results in less noise. Other characteristics, such as diameter, can also be modified to adjust the sound. The tubes illustrated in FIGS. 11A-C provide a range of noise attenuating intakes suitable for the user. In some instances a longer silencer with increased noise attenuation is desired, whereas for another user the option of a short tube with slightly louder noise output is desired for convenience and size purposes over the increase of noise generated from the noise. The option of offering ranges of intake tubes with varying sound attenuation properties is beneficial for users of PAP systems and other ventilation devices.

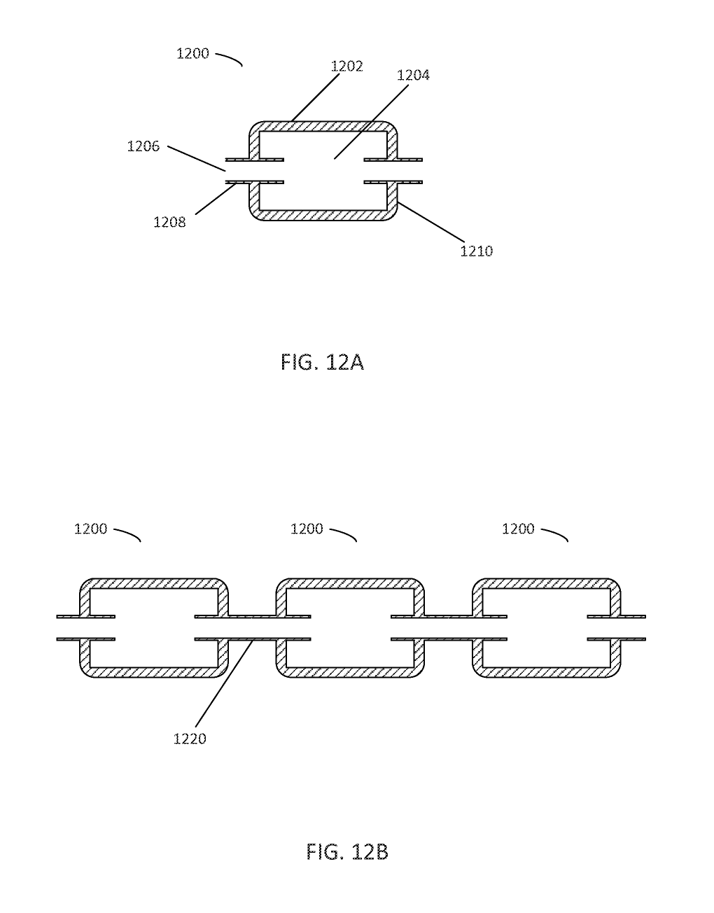

In addition to the intake tubes illustrated in FIGS. 11A-C, various expansion chamber units 1200 may also be linked together and form a flexible length as shown in FIGS. 12A-B. Expansion chamber unit 1200 has a housing 1202 with an interior expansion chamber 1204 that is proportionally defined by inlet port 1206 its cross-section, and length 1208 and wherein the inlet port 1206 is also proportionally defined by interior expansion chamber 1204. To help the linking expansion chamber units be more flexible when connected in series a portion of sidewall 1210 may be flexible near and around inlet port 1206, which helps the 1200 units hinge and flex about the inlet ports. Similar to FIGS. 11A-C these expansion chamber units may be of varying length. In some versions the expansion chamber units may include noise attenuating deflectors and/or materials such as porous material.

The systems and methods described herein are not exclusive to CPAP devices, but rather any device requiring flow to be generated and particularly pressurized flow. For example, the systems and methods described herein may also be adapted and applied to other PAP apparatuses, systems, and methods, including, but not limited to, automatic positive airway pressure devices [APAP], variable positive airway pressure devices [VPAP], bi-level positive airway pressure devices [BPAP], and related apparatuses, systems, and methods.

In certain approaches, the sound levels of the PAP systems and devices described herein range from less than 31 dBA, less than 30 dBA, less than 29 dBA, less than 26 dBA, and even less than 26 dBA.

In certain approaches, the systems and devices described herein may be less than 3 feet, less than 2 feet and even less than 1 foot in length. Though the system may be longer than 3 feet in length it is generally understood that shorter lengths are preferred.

It is contemplated that electronic circuitry, power supplies, processors, and other electronic components may be disposed outside of the tube containing the blower. Electrical leads/power connections may connect the external electronic components to the blower and screen contained within or on the CPAP in a hose system. In certain approaches, batteries may be used to power a PAP device to provide improved portability and ease of use.

In some embodiments the externally connected electrical components may be housed in a separate unit that is attached to a user/person's body via a strap, hook and loop system (such as Velcro®), inserted into pockets, glued, stapled, magnetically connected and so forth. These attachment devices may be used to attach, hang or otherwise dispose the external system to locations on or off the body or clothing of a user that is desired.

In some embodiments, external electrical components may be in wireless communication. Power sources may also be wireless and operate on principles such as inductive charging or powering.

The various components of PAP tube/hose provided herein may be physically coupled (e.g., wired) or wireless coupled. For example, a sensor may be wirelessly coupled to control circuitry in the expansion chamber via Bluetooth, Wi-Fi, near field communication, ZigBee, DECT, or other appropriate wireless protocol.

A processor may receive an input signal from the sensor. The processor may then process the signal according to software instructions stored in memory on a device such as smartphone, computer or remote control device. The processor may electronically store the signal or the processed signal as data in storage. The processor may send a control signal to flow generator/blower 614 to apply or adjust the air delivered by flow generator 614. In certain approaches, the processor provides a signal to display a value, instruction, or indicator related to the input signal at visual display on the smartphone, computer, remote control or other display either attached or remote from the PAP tube device. Additionally or alternatively, the processor may provide an audio signal, such as data, instructions, alarm, or indicator at audio output. In certain approaches, the processor may send or receive data, signals, indicators, instructions, or other information through remote network interface. For example, a PAP apparatus may communicate with an external electronic device, such as a phone, computer, or server. A remote network interface may be a transmitter, receiver, transceiver, antenna, communication port, or any other appropriate interface for communication with other systems and devices.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention.

1. A CPAP system comprising:
a tube having at least one expansion section; and
a gas flow generator disposed in a portion of the expansion section, wherein the gas flow generator comprises a motor to drive an impellor whereby a positive air pressure and flow are generated.

2. The system of claim 1, further comprising a coupling device along a downstream portion of the tube from the expansion section that detachably couples with a mask.

3. The system of claim 1, further comprising a display screen on a portion of the external surface of the expansion section of the tube.

4. The system of claim 1, wherein a power supply cord runs along the length of an upstream portion of the tube to the expansion section.

5. The system of claim 1, wherein the expansion section is an acoustic reduction chamber.

6. The system of claim 1, further comprising a second expansion section, configured to function as an acoustic reduction chamber.

7. The system of claim 6, further including noise deflectors placed within the second expansion section.

8. The system of claim 6, wherein noise reduction attenuating material is placed within the second expansion section.

9. The system of claim 6, wherein a portion of the electronics driving the motor are placed within the second expansion section.

10. The system of claim 1, wherein the blower is configured to produce pressures ranging from 2-40 cm H₂O.

11. The system of claim 1, wherein the tube is comprised of a plurality of detachable sections.

12. The system of claim 1, further comprising at least one of a circuit board, sensor, or power supply component disposed within the expansion section.

13. The system of claim 1, wherein electronic circuitry, processors or other electronic components are disposed outside of the tube.

14. The system of claim 12, wherein an electrical communication and power source connects the external electronic circuitry, processes, or electronic components with the blower.

15. The system of claim 12, wherein the electric circuitry, processors or other electronic components and/or the power supply and/or battery are attached to the mask or to a user.

16. An elongated CPAP system comprising:
a tube having an inlet port, an outlet, and an expansion section, and wherein the expansion section is positioned between the inlet port and the outlet; and
a gas flow generator positioned in the expansion section, wherein the gas flow generator generates positive gas pressure and flow through the outlet.

17. The system of claim 16, further comprising at least detachable attenuating noise tube section, configured to reduce the noise attenuated from the system.

18. The attenuating noise tube section of claim 17, further including an inlet that expands into a larger volume disposed within the attenuating noise tube section.

19. The attenuating noise tube section of claim 17, further including circuitous flow path within the attenuating noise tube section.

20. The system of claim 16, further comprising a separated user interface that remotely communicates with the blower.

21. The system of claim 16, further including at least one sensor for detecting flow rates connected to a flow port that is disposed downstream from the blower.

22. A portable and elongated CPAP system comprising:
an expansion tube section having a blower comprised of a motor and impellor disposed therein;
at least two detachable tube sections for attaching to an inlet and outlet port of the expansion tube section, wherein one of the detachable tube sections has acoustically designed inlet port that forms a low pass frequency filter with an expansion chamber disposed in the expansion tube section.
